(19) United States
(12) Patent Application Publication
(10) Pub. No.: US 2019/0274659 A1
(43) Pub. Date: Sep. 12, 2019

(54) SYSTEM AND METHOD FOR SPINAL IMAGING

(71) Applicant: Mayo Foundation for Medical Education and Research, Rochester, MN (US)

(72) Inventors: Neil G. Feinglass, Jacksonville, FL (US); Charles J. Bruce, Ponte Verda, FL (US); Christopher B. Robards, Jacksonville, FL (US)

(21) Appl. No.: 16/347,950

(22) PCT Filed: Nov. 9, 2017
(86) PCT No.: PCT/US2017/060909

§ 371 Date: May 7, 2019

Related U.S. Application Data
(60) Provisional application No. 62/419,545, filed on Nov. 9, 2016.

(51) Int. Cl. A61B 8/12; A61B 8/06; A61B 8/08; A61B 8/00; G01S 15/89; G01S 7/52

(52) U.S. Cl. CPC A61B 8/12; A61B 8/06; A61B 8/0875; A61B 5/026; G01S 15/8915; G01S 7/52079; A61B 8/4488

(57) ABSTRACT

A system and method are provided for imaging the spinal anatomy and cord blood flow of a subject. A device for use with an ultrasound console is provided and includes a catheter sized to be positioned within one of epidural and intrathecal space of the subject. The device also includes a plurality of imaging transducer elements spaced apart along a length of the catheter and a tether coupled to a proximal end of the catheter and configured to be coupled to the ultrasound console. The plurality of imaging transducer elements are configured to be controlled by the ultrasound console through the tether.

### CROSS-REFERENCE TO RELATED APPLICATIONS

This application is based on, claims priority to, and incorporates herein by reference in its entirety, U.S. Provisional Patent Application Ser. No. 62/419,545, filed on Nov. 9, 2016.

### BACKGROUND OF THE INVENTION

The present invention relates to systems and methods for imaging and, more particularly, systems and methods for imaging spinal anatomy and blood supply.

Imaging of the spine and its vascular supply allows for monitoring spinal cord anatomical changes in addition to assessing spinal cord blood supply. Recently, transesophageal echocardiography (TEE) has been used to produce two-dimensional (2D) and three-dimensional (3D) images of the spine. For example, a TEE probe is positioned inside an anesthetized patient and spinal structures are identified, enabling real-time monitoring of cord blood flow and anatomical structures.

TEE imaging thus provides a means for observing anatomical structures and physiological events during surgical interventions. However, such a system requires the patient to be anesthetized during the process, in which the TEE probe is inserted through the esophagus and positioned precisely to allow for optimal intervertebral disc alignment. Additionally, the large size of the TEE probe allows for minimal variance when positioning the device. Thus, TEE as a reliable and reproducible monitor of spinal structures has not expanded as a routine clinical monitor. In addition, generally only the thoracic portion of the spine is available for insonation using such procedures.

It is also possible to image the spinal cord vasculature using high-resolution and magnification angiography. For example, by injecting a radio-opaque contrast agent into the blood vessels, the vasculature can be visualized using X-ray imaging. While this technique is successful in imaging vasculature, the process is more invasive than desired and cannot be performed on patients who are critically ill or in the operating room.

Additionally, surface ultrasound systems have been used as an attempt to guide therapies of the spine. However, limitations of the patient's size and off-axis windows make continuous imaging challenging. Generally, this technique has only been demonstrated to see ligaments, tissue layers, and possible nerve roots. More specifically, the spinal cord and the vascular supply have not been effectively seen using this technique, though it may be possible for neonatal patients where imaging through bone is slightly less difficult.

Accordingly, it would be desirable to have a system and method for acquiring information about the spine and the vascular supply to the spinal cord and associated structures without the drawbacks described above.

### SUMMARY OF THE INVENTION

The present invention overcomes the aforementioned drawbacks by providing a system and method for 2D and 3D spinal imaging that utilizes a device inserted into the epidural or intrathecal space. An ultrasound imaging system, including the device, is provided to obtain real-time images of the spine as well as cord blood flow.

In accordance with one aspect, a device for use with an ultrasound console to image one of spinal anatomy and cord blood flow of a subject is disclosed. The device includes a catheter sized to be positioned within one of epidural and intrathecal space of the subject and a plurality of imaging transducer elements spaced apart along a length of the catheter. The device also includes a tether coupled to a proximal end of the catheter and configured to be coupled to the ultrasound console. The plurality of imaging transducer elements are configured to be controlled by the ultrasound console through the tether.

In a further aspect, a system for imaging spinal anatomy and cord blood flow of a subject is disclosed. The system includes a device comprising a catheter sized to be positioned within one of epidural and intrathecal space of the subject, a plurality of imaging transducer elements spaced along a length of the catheter, and a tether coupled to a proximal end of the catheter. The system also includes an ultrasound console configured to be coupled to the tether and to energize the plurality of transducer elements and receive a set of reflected signals from the plurality of transducer elements. The ultrasound console includes a display system and is configured to produce an image based on the reflected signals on the display system.

In yet a further aspect, a method for spinal imaging of a subject is provided. The method includes introducing a catheter including a plurality of imaging transducer elements spaced along a length of the catheter into one of an epidural and intrathecal space of the subject and positioning the catheter at a target region within one of the epidural and intrathecal space. The method also includes setting one or more parameters of the catheter to capture image data of at least a portion of the target region and acquiring the image data of the at least portion of the target region. The method further includes generating output including one of an image of the at least portion of the target region and an output variable related to the target region.

The foregoing and other aspects and advantages of the invention will appear from the following description. In the description, reference is made to the accompanying drawings which form a part hereof, and in which there is shown by way of illustration at least one embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic illustration of an ultrasound imaging system.

FIG. 2 is a schematic illustration of a device, in accordance with the present disclosure, including a catheter with a plurality of microcrystal imaging elements.

FIG. 3 is a schematic illustration of a catheter, in accordance with the present disclosure.

FIG. 4 is a flow chart illustrating a method for spinal imaging, in accordance with the present disclosure.

FIG. 5 is a schematic illustration of an embodiment of the present disclosure, detailing the positioning of a device relative to a spine of a subject.

FIGS. 6A-6C are views of method steps for inserting a device, in accordance with the present disclosure, relative to a spine of a subject.

### DETAILED DESCRIPTION

As will be described, systems and methods for two-dimensional (2D) and three-dimensional (3D) imaging of locations along the neuraxis are provided herein using a catheter placed in the epidural space or intrathecal space of a subject. An operator, such as a surgeon or medical professional, can introduce the catheter into the epidural or intrathecal space via needle introduction, and position the catheter so that image data received via the catheter detail desired anatomical features. The catheter includes multiple locations along its length capable of acquiring image data and can be coupled to an imaging console that controls ultrasound output and image generation.

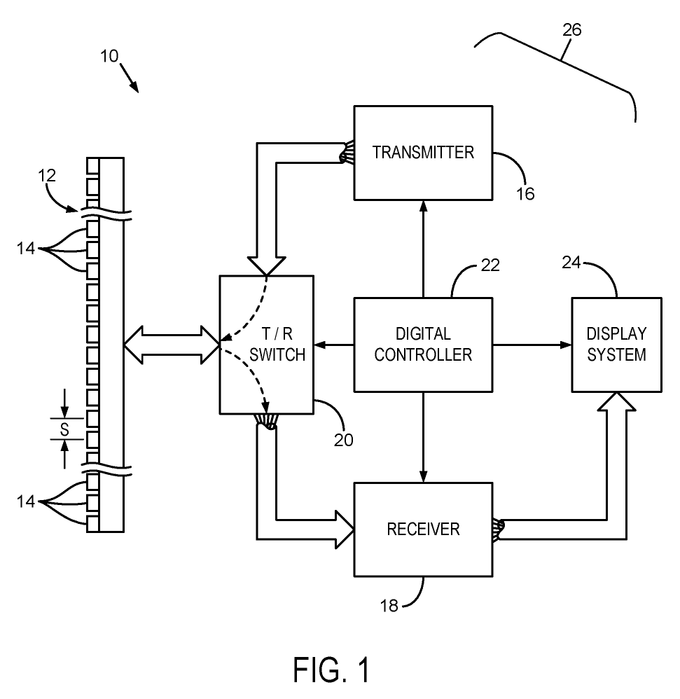

FIG. 1 illustrates an example motion imaging or ultrasound imaging system 10. As shown in FIG. 1, the system 10 includes a transducer array 12 (such as a linear array transducer) including plurality of transducer elements 14, a transmitter 16, a receiver 18, a switch mechanism 20, a digital controller 22 and a display system 24. In some applications, the transmitter 16, the receiver 18, the switches 20, the controller 22, and/or the display system 24 may be considered an echo console 26 or imaging console, where the transducer array 12 (and/or other transducers) can be removably coupled to and controlled by the echo console 26.

Generally, the plurality of transducer elements 14 of the transducer array 12 can be separately driven. For example, each transducer element 14 can be separately energized by the transmitter 16 to produce a burst of ultrasonic energy (an “echo signal”), which can be directed toward a target region of a subject under study. Some of this emitted ultrasonic energy can reflect off objects within the target region back toward the transducer array 12 and, when received by a transducer element 14, can be converted to an electrical signal. Each converted electrical signal is then applied separately to the receiver 18 through the switch mechanism 20. The transmitter 16, the receiver 18, and the switch mechanism 20 can be controlled by the digital controller 22 responsive to commands or input from a user (for example, from a computer console or other user interface, not shown).

More specifically, to acquire a series of echo signals, a plurality of switches in the switch mechanism 20 are first set to their transmit position, directing the transmitter 16 to momentarily energize each transducer element 14 to output an echo signal. The switches 20 are then set to their receive position and reflected echo signals received by each transducer element 14 are applied to the receiver 18. The separate echo signals from each transducer element 14 are combined in the receiver 18 to produce a single echo signal that is employed by the controller 22 to produce a line in an image, for example, displayed on the display system 24.

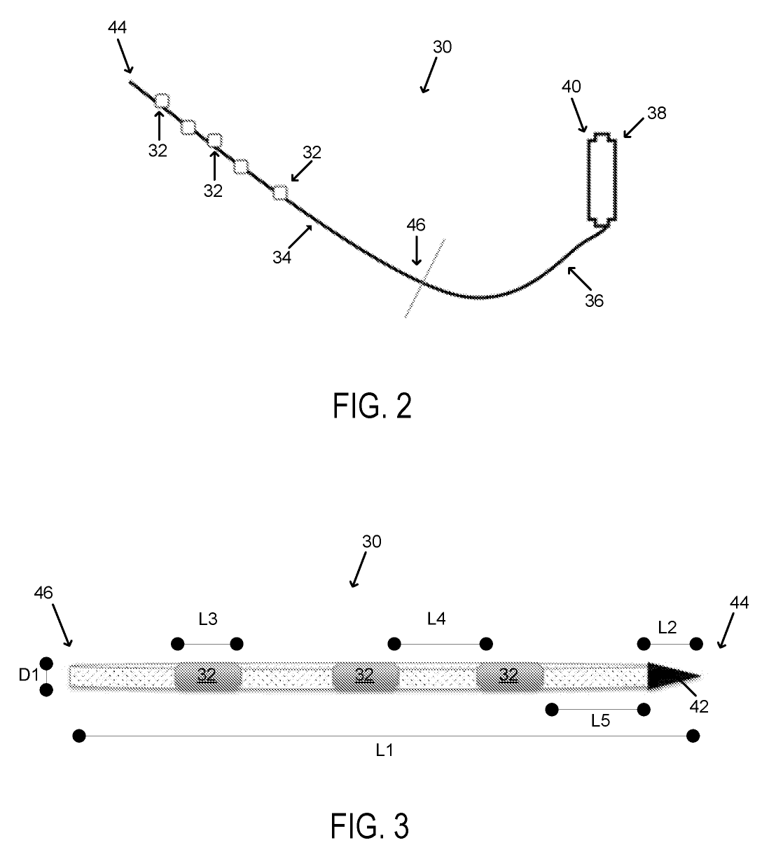

In some embodiments, the above-described echo console 26 (that is, the transmitter 16, the receiver 18, the switch mechanism 20, the controller 22, and/or the display system 24) can be integrated with or coupled to a device 30, as shown in FIG. 2. For example, the device 30 can be used with the echo console 26 or other suitable imaging systems to obtain image data for 2D and/or 3D images along a subject's neuraxis. More specifically, the device 30 can be sized for insertion into the epidural or intrathecal space (e.g., along a subject's spine) to allow for ultrasonic imaging of, for example, the spinal anatomy and blood supply.

As shown in FIG. 2, the device 30 can include an array of transducer elements 32, a catheter 34, a tether 36, and a connector 38. The connector 38, located at a proximal end 40 of the device 30, can be used to couple the device 30 to the echo console 26 for operation similar to that described above with respect to the transducer array 12. More specifically, the transducer elements 32 can be controlled by the ultrasound console 26 through the tether 36. In other words, the device 30 can be coupled to the echo console 26 via the connector 38 (such as a plug and play type connector), and the echo console 26 can control the device 30 to energize the transducer elements 32, receive returning echo signals, and produce image data. The image data can be used to render 2D and/or 3D images as well as generate variables of blood flow in real time.

As shown in FIG. 2, the array of transducer elements 32 can include a plurality of micro piezoelectric crystals (“microcrystals”) spaced apart along a length of the catheter 34 to generate one or more imaging beams that insonate the local anatomy. As such, the transducer elements 32 can be used to generate image data along a length of the neuraxis. More specifically, by energizing groups of transducer elements 32 along the device 30 at various locations relative to the neuraxis, a desired set of real-time images can be obtained. In some embodiments, the microcrystals 32 are exposed on a surface of the catheter 34. In other embodiments, the catheter 34 can include a sheath (not shown), and the sheath can include acoustic windows capable of accommodating internal radial or phased array transducers 32. Additionally, while five transducer elements 32 are illustrated in FIG. 2, it is within the scope of this disclosure to include a device 30 having two, three, four, or more transducer elements 32 to best suit a desired application.

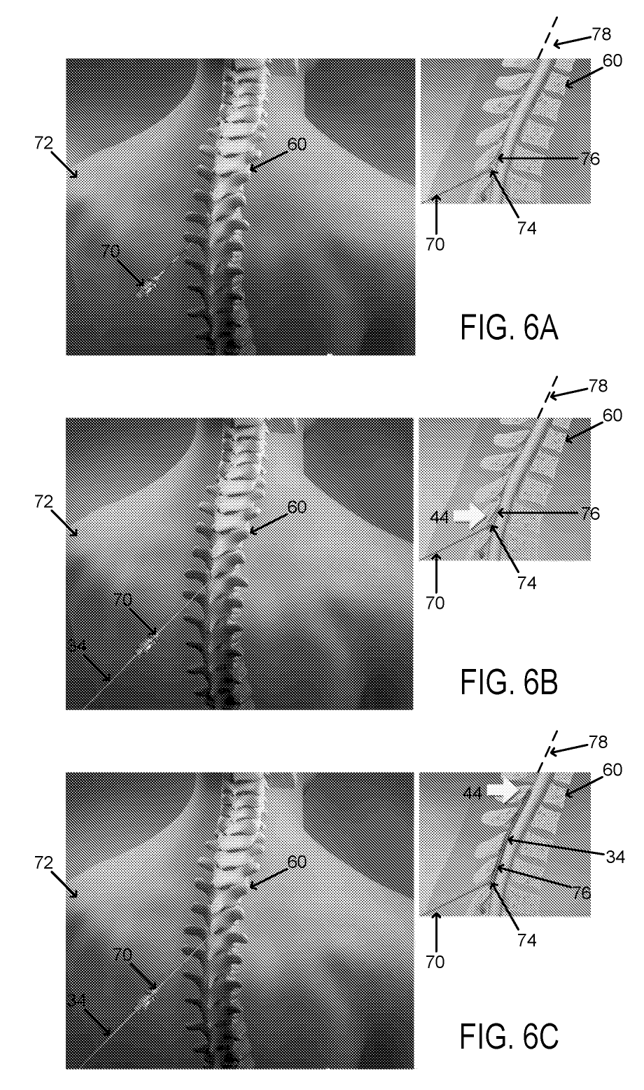

For example, FIG. 3 illustrates a catheter 34, for use in some applications, including three transducer elements 32. Generally, the catheter 34 can be sized to permit navigation through vascular structures without damaging local tissue or neuraxial layers of tissue. According to one embodiment, the catheter 34 can have a length L1 of about 150 millimeters (mm) and an inner diameter D1 between about 3.0 Fr (French gauge) to about 3.5 Fr, or to about 5.0 Fr (in some embodiments, this diameter range may also apply to an outer diameter of the catheter 34; however, other outer diameter ranges, or inner diameter ranges, may be contemplated within the scope of this disclosure). Additionally, to allow for precise localization of the catheter 34, a length of the catheter 24 (and/or other portions of the device 30) can be steerable and substantially stiff and rigid, yet flexible enough to bend during insertion, as shown in FIGS. 6B-6C. For example, in some embodiments, the catheter 34 can include a steerable sheath; however, in other embodiments, the catheter 34 itself may be steerable. As such, the catheter 34 can permit seamless and frictionless maneuvering and exchange to avoid trauma of the surrounding anatomy. Furthermore, as shown in FIG. 3, the catheter 34 can include a distal tip 42 at its distal end 44, such as a soft rounded tip, that is about 10 mm in length (L2) to further enhance seamless movement of the catheter 34.

As shown in FIG. 3, the three transducer elements 32 (or microcrystals) can be spaced apart along the length of the catheter 34. According to one embodiment, the microcrystals 32 can be about 0.5 centimeters (cm) to 1.0 cm in length (L3) and can be spaced apart about 2 cm to about 3 cm (L4) from each other. The distal-most microcrystal 32 can be about 1.0 cm to about 1.5 cm (L5) from the distal tip 42. Each microcrystal 32 can be configured to emit a particular image beam size and radius, which may be fixed or modifiable. For example, in some embodiments, the microcrystals 32 can be configured for a 10-mm to 30-mm imaging radius. Additionally, the microcrystals 32 can completely or only partially surround the diameter of the catheter 34. As such, each microcrystal 32 can emit an image beam that extends outward from the catheter 34, for example, 360 degrees, 270 degrees, 180 degrees, 90 degrees, or another suitable degree range. In some embodiments, radial transducer elements 32 are used (that is, capable of transmitting in a single direction, though larger diameter imaging beams may still be created by rotating the elements 32 and/or the catheter 34). In other embodiments, phased array elements 32 are used, in which an imaging beam around a diameter or partial diameter of the catheter 34 can be created without needing to rotate the catheter 34.

Additionally, the microcrystals 32 can be biologically safe when used within the human body. As such, the microcrystals 32 can be selected and/or operated in a way so that the heat generated by the microcrystals 32 during use of the device 30 can be below a predetermined threshold. For example, this threshold can correlate to a level that ensures that the clinical viability of the neural and spinal tissues surrounding the catheter 34 are not afflicted during use of the device 30.

Referring back to FIG. 2, the tether 36 of the device 30 can be coupled to a proximal end 46 of the catheter 34, and can be further coupled to the connector 38. The tether 36 can thus provide freedom of movement between the catheter 34 and an echo console 26, and can also facilitate the necessary connections for communication (e.g., signal transfer) between the catheter 34 and the echo console 26. In some embodiments, the device 30 can include multiple tethers 36 (e.g., a dedicated tether for each microcrystal 32). Generally, the device 30 can be sized so that the tether(s) 36 remains outside of a subject when the catheter 34 is positioned for imaging. As such, the device 30 can be split into an operational section (including the catheter 34) and an external section (including the tether(s) 36 and the connector 38). In some embodiments, the location of the separation between the operational section and the external section may be clearly marked to ensure that bacteria are not introduced to the subject during use of the device 30. However, in other embodiments, the tether(s) 36 may be sterile to reduce the risk of bacteria introduction via the device 30 during use.

Additionally, in some embodiments, the device 30 can be electrically grounded (that is, can include an electrical ground connection, not shown) to protect it from micro currents and electrical leakage during use. By insuring the electrical stability of the device 30, the safety and signal fidelity associated with the device 30 are inherently improved. Additionally, grounding the device 30 allows for insulation from commonly used devices in an operating room or surrounding area, such as Bovie electrosurgery devices, electrocardiographs, twitch monitors, and the like. More specifically, grounding the device 30 reduces the risk of such devices interfering with the signals from both the transmitters and the microcrystals 32.

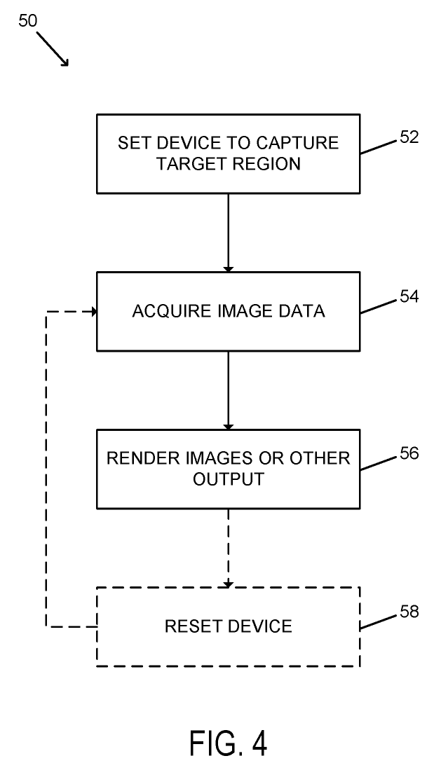

FIG. 4 illustrates a method 50 of using the device 30. Generally, as shown in FIG. 4, the method 50 includes setting the device 30 to capture at least a portion of a target region at step 52, acquiring image data at step 54, and rendering or generating images or output variables at step 56. Optionally, the method 50 also includes resetting the device 30 to capture a new target region or a different portion of the target region at step 58 and repeating steps 54 and 56.

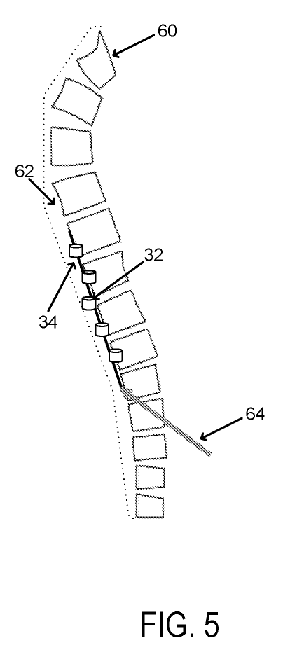

Referring to step 52, initially setting the device 30 to capture a target region can include positioning the device 30 as well as setting echo signal parameters to create an imaging region. Notably, the imaging region can include part of or the entire target region. For example, at this step, the catheter 34 can be inserted along the neuraxial space (such as the epidural space or the intrathecal space) near or at the target region. In some cases, the catheter 34 can be threaded upward from a lower site to the target region. FIG. 5 illustrates an example placement of the device 30 along a subject's spine 60, where the catheter 34 is positioned to reside within the spinal canal in the epidural, subarachnoid, or intrathecal space 62 along a target region. Arrow 64 illustrates an insertion route, with an insertion point into the intrathecal space 62 near the target region. In some applications, a hollow needle may be inserted through the insertion route to facilitate positioning the catheter 34.

More specifically, the catheter 34 can be inserted at a desired location by a surgeon, anesthesiologist, or medical professional under direct guidance (e.g., direct placement adjacent to the target region during an open operation or surgical field) or through needle introduction (e.g., similar to that of an epidural procedure). As a non-limiting example, an 18 gauge needle system may be used; however a 14 gauge system or other sizes may also be desirable in certain applications. As such, the diameter and length of the catheter 32 may be small enough to allow for insertion through clinically used needle systems.

FIG. 6A-6C illustrate an example needle introduction. As shown in FIG. 6A, a hollow needle 70 is inserted into a subject 72 (e.g., with the assistance of a stylet, not shown) at a desired location until a tip 74 of the needle 70 is adjacent the epidural space 76 (or intrathecal space). The catheter 34 can then be routed through the needle 70, as shown in FIG. 6B, and further inserted past the needle tip 74 so that the catheter 34 travels along the neuraxis 78 a desired length to capture a target region, as shown in FIG. 6C. In some applications, a break-away needle, such as a Touhy needle (e.g., used for epidural anesthesia insertion and related applications), can be used. As such, the above insertion method may be similar to methods used for placing neuromodulation leads for pain management or bladder spasticity control. Additionally, the use of a needle 70 to introduce the catheter 34 into the epidural space 76 allows for minimal tissue disruption, for example, compared to insertion via a trocar.

While the above example describes catheter insertion and placement along a length of the neuraxis, it should be noted that step 52 can further include rotating the catheter 34 to ensure the microcrystals 32 are properly positioned relative to the target region (or a portion thereof). Furthermore, with respect to step 52, echo signal parameters can be set to create an imaging region that matches the target region (or a portion thereof). More specifically, echo signal parameters, such as output frequency, power output, focusing, gain, time-gain compensation (TGC), and/or other parameters, can be selected based on the target region to be imaged (e.g., depth, area, etc.) and the desired output. For example, echo parameters can be set for direct imaging of vascular structures, such as anterior and posterior spinal arteries, as well as imaging characteristics of the cord and cerebral spinal fluid. In addition, echo parameters can be set to obtain color images for determining Doppler measurements of the vessels (e.g., color flow, continuous wave Doppler, and pulsed wave Doppler modalities can be used to measure blood flow). Additionally, echo parameters may be selected based on a desired object to be imaged within the target region, such as spinal vasculature, tumors, spinal cord tissue, blood clots, echo-visible agents, or others. In some embodiments, catheter rotation and echo parameters can be set and/or adjusted by the controller 22 of the echo console 26 (e.g., via user inputs to the echo console 26).

Furthermore, generally, the number and placement of microcrystal transducer elements 32 along the catheter 34 can be selected, prior to placement, based on a length and/or a region to be imaged. For example, the microcrystals 32 can be arranged for imaging one or more anatomical regions within the spine, such as one or more of the cervical, thoracic, and lumbar regions. As a result, due to the multiple transducer elements 32, the device 30 can be configured to visualize some or all regions of the neuraxis without requiring repositioning of the device 30. Accordingly, the plurality of spaced-apart microcrystal transducer elements 32 provides for a greater imaging area than what has been previously instituted, as imaging information can be received along a length of the catheter 34 (or the entirety of the catheter 34), rather than being focused primarily at a distal tip of the device 30.

Referring now to step 54, image data is acquired using the positioned catheter 34. More specifically, using an imaging system or echo console 26 coupled to the catheter 34, the plurality of spaced-apart microcrystal transducer elements 32 are controlled to insonate the local anatomy around the catheter 34 (that is, transmit ultrasound energy and receive subsequent reflected signals) based on the set echo parameters. As such, the image data may be considered ultrasound image data.

At step 56, the acquired image data may be processed to generate 2D images, 3D images, and/or one or more output variables (such as, but not limited to, measurements of blood flow, Doppler signals, color flow Doppler, tissue motion, cell viability, post-surgical changes, and/or cell deposition). For example, these images or output variables can be displayed to an operator via the display system 24 of the echo console 26. Additionally, in some applications, such images and/or output variables can be generated in real time or near real time. For example, the device 30 can be used for direct observation of the target region during surgical procedures by generating real-time Doppler measurements of blood flow. Such real-time imaging during a surgical procedure cannot be done using previous techniques (e.g., due to challenges presented by anatomical variability of spinal vasculature or challenges of being in a surgical suite or ICU and not a dedicated imaging room). In a further example, the above-described Doppler capabilities can be used to detect low-flow in spinal arteries that run perpendicular to the imaging beams. In some embodiments, image processing can be executed by the controller 22 of the echo console 26, for example, in response to user inputs.

Additionally or alternatively, in some applications, a report, including images, output variables, and/or other data, may be generated and/or saved for later display. For example, in some applications, imaging can be done through the perioperative period (e.g., in the ICU) and such images viewed later during surgery. Furthermore, imaging can be done post-operatively and compared to pre-operative or surgical images or data.

With respect to step 58, optionally, the imaging region can be modified, for example to capture other portions of the target region or a new target region. For example, in some applications, the geometry of the image beam produced by the microcrystal transducer elements 32 can be modified remotely by a user at the echo console 26 or other user interface. For example, in some embodiments, the image beam can be rotated by adjusting the echo parameters (and/or rotating the catheter 34). In another example, in some embodiments, a depth of the image beam can be modified by adjusting a frequency parameter of the echo signal. Additionally, other types of modifications are contemplated within the scope of this disclosure. Accordingly, the device 30, connected to a console 26, is capable of producing an imaging set with alternative anatomical features without repositioning the device 30 superiorly or inferiorly within the dural space.

In light of the above, some embodiments provide a device 30 including an ultrasound catheter 34 to be used in the epidural or intrathecal space of a subject and having multiple transducer elements 32 positioned along its length. The device 30 may be used with an imaging console 26 to obtain 2D or 3D images of a surrounding target region and/or one or more output variables related to the target region.

Furthermore, the present device 30 can additionally or alternatively be used for other types of monitoring or therapeutic uses. For example, in some embodiments, a sheath of the catheter 34 can be hollow and can include one or more therapeutic channels or ports. More specifically, the sheath can include one or more inlets at or adjacent the proximal end 40 and one or more outlets at or adjacent the distal end 44. As such, the catheter 34 can include one or more channels through the hollow sheath configured to accommodate one or more tools or to deliver one or more agents from the proximal end 40 to the distal end 42. Such tools can be used with the device 30 for, for example, microsurgery and/or endoscopic procedures. Another example tool includes an optical fiber capable of acquiring video images from the distal end 42. Further example tools can include sensors, for example, to enable oximetry monitoring or neural signal monitoring via the device 30. The channels can also be used to introduce therapeutic modalities, such as stem cell introduction, echo visible agents, chemotherapy agents, regenerative therapy agents, or other agents.

Accordingly, the device 30 may be used in the epidural or intrathecal space of a subject to obtain real-time images of the spine (e.g., spinal ultrasound) and/or cord blood flow, for example, before, during, or after surgery. The device 30 may also be used, for example, to monitor spinal regions (e.g., post-operatively and/or in an ICU setting for subjects with paraplegia concerns). Also, the device 30 may be used to image and/or treat spinal masses, infarcts, arteriovenous malformations (AVMs), hematomas, ailments of the brainstem, high cervical lesions or other structures or ailments. Additionally, the device 30 may assist with procedures of the aorta (such as by helping visualize anatomy and monitor blood flow to nerves and cord during dissections, thoracic aneurysm procedures, endovascular aneurysm repair (EVAR) procedures, etc.). Furthermore, the device 30 can be used to administer medications and/or stem cells in the epidural or intrathecal space, or for intrathecal draining, monitoring, or administrations, or for monitoring neural cell viability, stem cell administration, spinal medication treatments, metastasis to the spine and neural structures, and/or degenerative changes in the spine. Additionally, the device 30 may be used at any location along the neuraxis, and may further be used intracranially (e.g., in the ventricular system).

While the invention is described through the above-described exemplary embodiments, it will be understood by those of ordinary skill in the art that modifications to, and variations of, the illustrated embodiments may be made without departing from the inventive concepts disclosed herein. Moreover, while the preferred embodiments are described in connection with various illustrative data structures, one skilled in the art will recognize that the system may be embodied using a variety of data structures. Furthermore, disclosed aspects, or portions of these aspects, may be combined in ways not specifically listed above. Accordingly, it is felt therefore that the scope of protection provided by this patent should not be viewed as limited by the above description, but rather should only be limited by the scope of the below claims.

1. A device for use with an ultrasound console to image one of spinal anatomy and cord blood flow of a subject, the device comprising
a catheter sized to be positioned within one of epidural and intrathecal space of the subject;
a plurality of imaging transducer elements spaced apart along a length of the catheter; and
a tether coupled to a proximal end of the catheter and configured to be coupled to the ultrasound console, the plurality of imaging transducer elements configured to be controlled by the ultrasound console through the tether.

2. The device of claim 1, wherein the plurality of imaging transducer elements includes a plurality of micro piezoelectric crystals.

3. The device of claim 1 and further comprising an electrical ground connection coupled to the device.

4. The device of claim 1, wherein the plurality of imaging transducer elements are configured to rotate to acquire information in a plurality of directions.

5. The device of claim 1, wherein the plurality of imaging transducer elements includes phased array elements.

6. The device of claim 1, wherein the catheter includes a sheath.

7. The device of claim 6, wherein the sheath includes at least one channel.

8. The device of claim 1, wherein the plurality of imaging transducer elements are positioned inside the sheath and the sheath includes at least one acoustic window.

9. The device of claim 1 and further comprising a connector coupled to a proximal end of the tether and configured to connect the tether to the ultrasound console.

10. A system for imaging spinal anatomy and cord blood flow of a subject, the system comprising
a device comprising:
a catheter sized to be positioned within one of epidural and intrathecal space of the subject,
a plurality of imaging transducer elements spaced along a length of the catheter, and
a tether coupled to a proximal end of the catheter; and
an ultrasound console configured to be coupled to the tether and to energize the plurality of imaging transducer elements and receive a set of reflected signals from the plurality of imaging transducer elements, the ultrasound console including a display system configured to produce an image based on the reflected signals on the display system.

11. The system of claim 10 and further comprising a needle system configured to guide the device to a location within one of the epidural and intrathecal spaces.

12. The system of claim 11, wherein the needle system is one of a 14 gauge system and an 18 gauge system.

13. The system of claim 11, wherein the needle system is a break-away type needle.

14. The system of claim 10 and further comprising a connector coupled to the tether and configured to connect the tether to the ultrasound console.

15. The system of claim 10, wherein the plurality of imaging transducer elements includes a plurality of micro piezoelectric crystals.

16. A method for spinal imaging of a subject, the method comprising the steps of:
a) introducing a catheter including a plurality of imaging transducer elements spaced along a length of the catheter into one of an epidural and intrathecal space of the subject;
b) positioning the catheter at a target region within one of the epidural and intrathecal space;
c) setting one or more parameters of the catheter to capture image data of at least a portion of the target region;
d) acquiring the image data of the at least portion of the target region; and
e) generating output including one of an image of the at least portion of the target region and an output variable related to the target region.

17. The method of claim 16, wherein the output variable includes a measurement of blood flow within the target region.

18. The method of claim 16, wherein step a) includes inserting a needle into the subject until a tip of the needle is adjacent one of the epidural and intrathecal space, and inserting the catheter through the needle.

19. The method of claim 16 and further comprising f) resetting the one or more parameters of the catheter to capture image data of at least another portion of the target region.

20. The method of claim 16, wherein step c) includes acquiring ultrasound image data by energizing the plurality of imaging transducer elements and receiving a set of reflected signals from the plurality of imaging transducer elements.
